(19) United States
(12) Patent Application Publication
(10) Pub. No.: US 2007/0160704 A1
(43) Pub. Date: Jul. 12, 2007

(54) Colonies of nostoc commune, methods for cultivating edible nostoc commune and edible nostoc commune formulations and their use for promoting health

(76) Inventors: Fan Lu, Clemmons, NC (US); Qiang Hu, Chandler, AZ (US); Zheng Yu Hu, Wu Chang City (CN); Yang Zhong, Wuchang City (CN)

Correspondence Address: KILPATRICK STOCKTON LLP, 1001 WEST FOURTH STREET, WINSTON-SALEM, NC, 27101, US

(21) Appl. No.: 10/587,424

(22) PCT Filed: Feb. 3, 2005
(86) PCT No.: PCT/US05/03314

§ 371 Date: Sep. 27, 2006

Related U.S. Application Data
(60) Provisional application No. 60/541,286, filed on Feb. 3, 2004.
(60) Provisional application No. 60/541,290, filed on Feb. 3, 2004.

(51) Int. Cl. A61K 35/74; C12N 1/20

(52) U.S. Cl. 424/780; 435/252.1

(57) ABSTRACT

Nostoc formulations, dietary supplements comprising the Nostoc formulations, and food products comprising Nostoc commune, (also known as Nostoc sphaericum, or Nostoc commune var. sphaericum) are disclosed. Nostoc formulations that may additionally comprise a medicinal composition are described. The present invention further relates to processes for producing these Nostoc formulations. In addition, the present invention relates to methods for promoting the health of an individual utilizing the Nostoc formulations, dietary supplements, food products and/or pharmacological compositions of the present invention. Moreover, this invention provides a method for cultivating Nostoc commune comprising (a). isolating and purifying Nostoc commune; (b). culturing the Nostoc commune; and (c). conditions suitable for optimal growth of Nostoc commune.

### STATEMENT OF RELATED APPLICATIONS

The present application claims priority under 35 USC §119 to provisional application 60/541,290 filed Feb. 3, 2004 and to provisional application 60/541,286 filed Feb. 3, 2004, the disclosure of each provisional application being hereby incorporated by reference.

### FIELD OF THE INVENTION

The present invention relates to isolated Nostoc commune cells and colonies; methods for their production; compositions, dietary supplements, pharmaceuticals, foods, and the like comprising Nostoc commune and their use in treating various medical conditions.

### BACKGROUND

Prokaryotic blue-green algae (cyanobacteria) are amongst the most primitive life forms on Earth. These prokaryotes share structural features with plants, such as having the ability to perform photosynthesis. Moreover, they share structural features with primitive bacteria in that they lack a cell wall. However, they share some characteristics with higher order organisms in the animal kingdom. One characteristic that they share is that they often contain complex sugars similar to glycogen on their cellular membrane. There is a great variety present in blue-green algae. For example, there are both edible and toxic species. Many species are adapted to the most extreme habitats on Earth, including hot springs, deep-sea vents, and polar ice habitats. One genus of blue-green algae, Nostoc, has been used for food for thousands of years. Indigenous populations in communities as diverse as the people of Japan and Hawaii, and those that occupy terrain where there are large freshwater lakes, including Lake Chad in Africa, Klamath Lake in North America, Lake Texcoco in Mexico, and Lake Titikaka in South America have long recognized the value and health benefits of blue-green algae in their diets.

Recent research has confirmed the beliefs of these indigenous populations in the value of ingesting blue green algae. In particular, this recent research has shown that there are health benefits to blue green algae in general, and to the Nostoc genus, in particular. One such benefit has been its use as a herbal medicine and also in perioperative care. (See Ang-Lee 2001). Other benefits include use as an anti-bacterial medicine, an anti-inflammation medication, use as an anti-carcinogen, an anti-viral medicine, and cholesterol lowering activity, and the like. (See Aki et al. 2000, Golaloti et al. 1995, Gonzalez et al. 1999, Smith et al. 1994, Murakami et al. 1997, Esser et al. 1999, Hayashi et al. 1996, and Knubel et al. 1990). Thus, it is desired that large quantities of the Nostoc genus be produced to take advantage of these health benefits. These large quantities can then be used as an addition to food products, or as an addition to a pharmaceutical composition or used as the main active ingredient in a pharmaceutical composition, a dietary supplement, as medication, or the like.

The cyanobacterium Nostoc commune, also known as Nostoc sphaericum, or Nostoc commune var. sphaericum, appears as spherical macroscopic colonies (“pearls”) in natural habitats. Nostoc commune (N. commune) is a filamentous, nitrogen-fixing cyanobacterium belonging to the family of Nostocaceae in the order of Nostocales (Komarek and Anagnostidis, 1989). In natural habitats, such as rice paddies, shallow streams, water ponds, and large open fields, N. commune can form spherical macroscopic colonies consisting of filaments embedded in a gelatinous matrix. The size of colonies ranges from tens of mm to tens of cm in diameter with the largest described being 2.6 kg wet weight (Dodds et al. 1995). The colonies range in color from yellow-green to red-brown, and dark green to black (Potts, 2000). The filaments are unbranched and largely twisted, and consist of mostly vegetative cells with a few heterocysts occurring in the middle of a filament.

Reproduction of N. commune takes place in four different ways, depending on environmental conditions: 1) single cells of N. commune fragmented from filaments can form new colonies; 2) akinete formation and germination, 3) hormogonia disperse and form new colonies; 4) large colonies can bud off to form separate colonies (Dodds, et al., 1995).

There are, however, drawbacks to current methods of producing large amounts of Nostoc. With the advent of modern agricultural techniques, man has been able to produce large amounts of grains and other agricultural products. However, there have been drawbacks associated with these techniques including the presence of run-off from fertilizers, pesticides, fungicides, insecticides, herbicides and other hazardous waste materials. Thus, one potential drawback to harvesting large quantities of blue green algae from its natural source, and Nostoc in particular, is the potential contamination that is present due to the run-off of these fertilizers, pesticides, fungicides, insecticides, herbicides, and other hazardous waste materials. Accordingly, it is desired to produce large amounts of blue green algae that are relatively free of these contaminants. Removing these potentially dangerous contaminants in the traditional manner has often been prohibitively expensive. Moreover, a recent study has shown that the presence of these contaminants may lead to the destruction of the Nostoc genus. (See Qiu et al. 2002).

When blue green algae is harvested from its natural sources, there are not only the above-mentioned man-made contaminants but there is also potential contamination from natural contaminants such as mud, sand, grass, or dirt. Ridding the blue green algae of these contaminants when done on a large scale adds prohibitively to the cost of large scale isolation. Other possible contaminants that preclude the formation of axenic colonies include bacteria, other algae, fungi, and other organisms. To rid the blue green algae of these contaminants when done on a large scale adds further costs to a large scale isolation process.

Another potential drawback of harvesting blue green algae from its natural source is that the blue green algal colonies are present in a great variety of sizes. When blue green algae is present in a variety of sizes, the production of axenic colonies of cyanobacteria can be prohibitively costly. One such factor that add to these costs is the removal of contaminants alluded to above. Thus, ideally, one would desire that the algal colonies be roughly uniform in size.

Another drawback to harvesting blue green algae from its natural source is that one is limited to particular seasons of the year when the blue green algae can be harvested. In particular, one is limited to harvesting the blue green algae during the spring and summer seasons. Thus, it is desired that one be able to harvest blue green algae throughout the year. It is with these considerations in mind that the present invention was developed.

### DESCRIPTION OF RELATED ART

To the inventors' knowledge, prior to the instant invention, large amounts of cultured axenic colonies of Nostoc genus have never been produced.

U.S. Pat. No. 6,667,171 describes an apparatus for removing carbon containing compounds from flowing gas streams using a plurality of photosynthetic microns including cyanobacteria. U.S. Pat. No. 6,579,741 discloses a method of culturing algae capable of producing large amounts of unsaturated fatty acids, an or phototrophic pigments and/or polysaccharides using a dome shaped, a conical shaped, or a cylindrical shaped apparatus. However, U.S. Pat. No. 6,579,741 suffers the drawback of producing a final solution that is rife with contaminants. Thus, because of the potential health benefits of cyanobacteria in general, the Nostoc genus in particular, and more particularly Nostoc commune, it is desired to produce large axenic quantities of these organisms without the presence of contaminants as is described above. It is with these considerations in mind that the instant invention was developed.

The Huang et al reference discloses suspensions of Nostoc. However, Huang et al. do not generate colonies of Nostoc and thus, the suspensions that they refer to are only for filament suspensions. In other words, Huang et al. fail to disclose any conditions that would allow the generation of colonies (either microcolonies or macrocolonies).

### BRIEF SUMMARY OF THE INVENTION

The present invention relates to isolated Nostoc commune cells, an isolated Nostoc commune strain and methods for mass cultivation of these isolated Nostoc commune cells and strains. More specifically, this invention relates to a method for cultivating axenic Nostoc commune. More particularly, this invention relates to a method for large-scale cultivation of axenic Nostoc commune.

Another embodiment of the present invention provides edible Nostoc commune formulations, dietary supplements, and food products (including medical foods) comprising the Nostoc commune formulations and/or dietary supplements. The present invention also provides pharmaceutical compositions comprising Nostoc commune.

An edible Nostoc commune formulation of the present invention optionally includes fresh biomass and/or dried powder of Nostoc commune. The Nostoc commune formulation may contain one or any combination of members from the group selected from proteins, fatty acids, amino acids, polysaccharides, vitamins, natural pigments, and minerals. The food product of the instant invention includes dissolving the dried powder of Nostoc into any drinkable liquid, or suspension of the powder into any liquid and/or solid.

A dietary supplement of the present invention comprises a Nostoc commune formulation of the present invention. The dietary supplement may further comprise one or any combination of ingredients such as herbals or herbal extracts, algal biomass or their extracts, fungal extracts, enzymes, fiber sources, minerals, vitamins and the like.

A food product of the present invention comprises a Nostoc commune formulation of the present invention and/or a dietary supplement of the present invention. The food product may further comprise one or any combination of ingredients such as herbals or herbal extracts, algal biomass and their extracts, fungal extracts, enzymes, a fiber source, minerals, vitamins and the like.

A pharmaceutical composition of the present invention comprises a Nostoc commune formulation of the present invention in a pharmacologically effective amount. The compositions may additionally comprise prescription medicines and/or non-prescription medicines. The combinations may advantageously produce one or more of the following effects:

1) additive and/or synergistic benefits;

2) reduction of the side effects and/or adverse effects associated with use of the prescription medicine in the absence of the Nostoc commune formulation; and/or

3) the ability to lower the dosage of the prescription medicine in comparison to the amount of prescription medicine needed in the absence of the soy formulation.

The Nostoc commune formulations, dietary supplements, food products and/or pharmaceutical compositions of the present invention may advantageously be utilized in methods for promoting the health of an individual.

The Nostoc commune formulations, dietary supplements, food products and pharmaceutical compositions of the present invention may also provide one or any combination of proteins, fatty acids, amino acids, polysaccharides, vitamins, natural pigments, and/or minerals. The proteins, fatty acids, amino acids, polysaccharides, vitamins, natural pigments, and/or minerals provided by the Nostoc commune formulations, dietary supplements, food products and pharmaceutical compositions of the present invention may provide numerous health benefits to an individual. The pharmaceutical composition of the present invention may further contain any suitable carriers, excipients, diluents, solvates, or other inert carriers.

Another embodiment of the present invention is a bioreactor that is designed for large scale cultivation of cyanobacteria or other bacterial species. The bioreactor will be described with reference to the drawings. Those of ordinary skill in the art will recognize that these drawings are merely illustrative of one embodiment of a bioreactor and are not to be construed as to limit the scope of this embodiment of the invention.

Further details and advantages of the present invention are provided in the following more detailed description.

### BRIEF DESCRIPTION OF THE SEVERAL VIEWS OF THE DRAWING

FIG. 1 is a 3-D view of one embodiment of a bioreactor, which includes a depiction of the growth columns.

FIG. 2 is a 3-D view of one embodiment of a bioreactor without the presence of the growth columns.

FIGS. 3A and 3B are a top view of the frame and a side view of the frame, respectively.

FIGS. 4A and 4B are a side view lengthwise view of the bioreactor and a side view widthwise of the bioreactor, respectively.

FIGS. 5A and 5B are a cross-sectional top view of the connecting braces K and the supporting rings L, and a blow-up sectional top view of the connecting braces K and the supporting rings L, respectively.

FIGS. 6A and 6B are a cross-sectional top view of one growth column with the underlying base and a 3-D view of a growth column J with the underlying base, respectively.

FIG. 7 is a 3-D view of a large tank that is supported on a frame wherein the frame is not depicted.

FIG. 8 is a 3-D view of a depicted frame which supports the large tank of FIG. 7.

FIG. 9 is a side view of a depicted frame which supports the large tank of FIG. 7.

FIG. 10 is a bottom view of a depicted frame which supports the large tank of figure

FIGS. 11A and 11B show the effect of light intensity (50, 100, or 200 μmol m⁻² s⁻¹) on the growth of Nostoc commune in Dry Weight (FIG. 11A) and in Chl-a (chloroform-a concentration (FIG. 11B)).

### DETAILED DESCRIPTION OF THE INVENTION

For the purposes of this specification, unless otherwise indicated, all numbers expressing quantities of ingredients, reaction conditions, and so forth used in the specification are to be understood as being modified in all instances by the term “about.” Accordingly, unless indicated to the contrary, the numerical parameters set forth in the following specification are approximations that can vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein. For example, a stated range of “1 to 10” should be considered to include any and all subranges between (and inclusive of) the minimum value of 1 and the maximum value of 10; that is, all subranges beginning with a minimum value of 1 or more, e.g. 1 to 6.1, and ending with a maximum value of 10 or less, e.g., 5.5 to 10. Additionally, any reference referred to as being “incorporated herein” is to be understood as being incorporated in its entirety.

It is further noted that, as used in this specification, the singular forms “a,” “an,” and “the” include plural referents unless expressly and unequivocally limited to one referent.

The present invention provides for a biologically pure culture of a cyanobacterium strain belonging to the genus Nostoc, wherein the culture comprises colonies of Nostoc wherein substantially all of said colonies have a diameter of between about 3 mm and about 5 mm and said culture is substantially free of contaminants, wherein the contaminants are selected from the group consisting of pesticides, fungicides, insecticides and herbicides. The preferred species of Nostoc is Nostoc commune.

By a biologically pure culture of cyanobacterium, it is meant that the culture is substantially free of contaminants such as pesticides, fungicides, insecticides and herbicides, substantially free of other contaminants including natural contaminants such as mud, sand, grass, and/or dirt, and substantially free of the presence of other organisms such as fungi, other algae, and the like. By substantially free of, it is meant that the Nostoc strain is present in an amount that is at least 90% pure, more preferably at least 95% pure and most preferably at least 99% pure.

By substantially all of the colonies having a diameter of about 3 mm to 5 mm, it is meant that at least 80% of all of the colonies are of about 3 mm to 5 mm in diameter, more preferably at least 85% of all of the colonies are of about 3 mm to 5 mm in diameter, and even more preferably at least 90% of the colonies are of about 3 mm to 5 mm in diameter and most preferably at least 99% of the colonies are of about 3 mm to 5 mm in diameter. Substantially all of the colonies have a size of smaller than 3 mm when the colonies are microcolonies. However, when the colonies are macrocolonies, diameters in excess of 3 mm can be obtained after several weeks, and after additional weeks of growth, colonies with diameters in excess of 10 mm can be obtained. In particular, after four weeks, about 80% of colonies reached a diameter of 10 mm.

The instant invention commences by growing colonies that have a diameter of about 0.1 mm. Thus, a colony has a size that is at least 0.1 mm, said colonies going though stages wherein the diameter is preferably at least 1 mm, subsequently through a size that is more preferably at least 2 mm and then through a stage wherein most of the colonies have a diameter that is most preferably between about 3 mm and 5 mm. At this stage, the number of colonies having a size of 3 mm to 5 mm tends to follow a gausian type distribution centered around 4 mm. In other words, most of the colonies are between 3 and 5 mm.

The uniformity of the colonies (wherein most of the colonies are between about 3 and 5 mm in diameter) is advantageous in that it reduces processing costs associated with having colonies that are of diverse sizes. When colonies are less than about 3 mm in size the quality of the colonies and any product derived from said colonies is inferior. When the colonies are greater than 5 mm in size, the colonies take too long to grow leading to higher costs. In nature, because colony growth is not well regulated, the inventors estimate that between 20˜30% of the colonies are between about 3 and 5 mm in diameter.

In another embodiment, the present invention provides a method of producing a biologically pure culture of a cyanobacterium strain belonging to the genus Nostoc, wherein said method comprises:

a) collecting a Nostoc colony from a natural source,

b) washing said Nostoc colony with a sterile medium,

c) crushing said Nostoc colony to generate a crushed Nostoc colony,

d) spreading said crushed Nostoc colony onto an agar plate,

e) illuminating said agar plate containing said Nostoc colony with fluorescent light,

i) transferring said Nostoc colony to a fresh agar plate,

g) repeating step f) from 1 to 3 times.

The above method generates a biologically pure culture of a Nostoc strain colonies. The preferred species of the above method is Nostoc commune, however it is understood that the above method can be employed with other Nostoc species and with other cyanobacteria.

The growth media/sterile media/media for agar plates are shown in Table 1. These media can be used to wash colonies and grow colonies, can be used to aid in the promotion of hormogonia or to aid in the formation of microcolonies or macrocolonies, can be used to resuspend colonies, and/or can be used in agar plates to promote colony growth. Preferably, the water used in the growth media is sterile so that the media is also sterile.

TABLE 1
Growth Medium: pH = 7.4 in all media

| Composition | Algaen I | | Algaen II | | Algaen III | |
|---|---|---|---|---|---|---|
| NaNO₃ | 1.5˜2.0 | g/L | 1.5˜2.0 | g/L | 1.5˜2.0 | g/L |
| MgSO₄ | 0.015˜0.045 | g/L | 0.075˜0.15 | g/L | 0.075˜0.15 | g/L |
| Na₂CO₃ | 0.02˜0.05 | g/L | 0.02˜0.05 | g/L | 0.02˜0.05 | g/L |
| CaCl₂•2H₂O | 0.36˜0.54 | g/L | 0.1˜0.18 | g/L | 0.36˜0.54 | g/L |
| EDTA | 0.001˜0.003 | g/L | 0.001˜0.003 | g/L | 0.001˜0.003 | g/L |
| K₂HPO₄ | 0.01˜0.02 | g/L | 0.04˜0.075 | g/L | 0.04˜0.075 | g/L |
| Citric Acid | 0.006˜0.01 | g/L | 0.006˜0.01 | g/L | 0.006˜0.01 | g/L |
| Fe(NH₄)₂Citrate | 0.006˜0.012 | g/L | 0.006˜0.012 | g/L | 0.006˜0.012 | g/L |
| Co(NO₃)₂•6H2O | 0.049˜0.085 | ppm | 0.049˜0.085 | ppm | 0.098˜0.17 | ppm |
| CuSO₄•5H₂O | 0.079˜0.15 | ppm | 0.079˜0.15 | ppm | 0.16˜0.3 | ppm |
| Na₂MoO₄ | 0.39˜0.82 | ppm | 0.39˜0.82 | ppm | 0.4˜1.2 | ppm |
| MnCl₂•4H₂O | 1.81˜3.62 | ppm | 1.81˜3.62 | ppm | 1.81˜3.62 | ppm |
| H₃BO₃ | 2.86˜5.67 | ppm | 2.86˜5.67 | ppm | 6.2˜12.4 | ppm |
| ZnSO₄•7H₂O | 0.05˜0.10 | ppm | 0.05˜0.10 | ppm | 0.05˜0.10 | ppm |

In particular, the above recited media can be advantageously used to generate ideal conditions for differentiation of colonies, for example, the Algaen III medium can be advantageously used to generate macrocolonies and the growth and reproduction of microcolonies can be advantageously performed in the Algaen I medium. In another example, the Algaen II medium can be used advantageously to wash macrocolonies and to produce hormogonia. Hormogonia are small, motile filaments formed by some cyanobacteria that may occur when the cyanobacteria are exposed to an environmental stress or may occur when placed in new media. Methods to stress the colonies include crushing colonies with mortar and pestle or other methods of grinding or pulverizing the colonies. Other stress methods to produce hormogonia are known to those of skill in the art and are included within the scope of this invention.

The preferred method of crushing microcolonies and/or macrocolonies is done with a mortar and pestle, however it is within the scope of the instant invention to employ other methods of reducing the size of colonies that are known to those of skill in the art including using grinders, other methods of pulverizing and the like.

Microcolonies and macrocolonies can be selectively generated by using different media and different strengths of fluorescent light. Microcolonies can be produced from hormogonia by suspending hormogonia in a sterile medium, preferable in a medium like Algaen I (as shown in Table 1) and spreading the suspended hormogonia on an agar plate comprised of a sterile medium such as Algaen I and containing agar in an amount that is between 0.5-50% by weight more preferably between 1-10% by weight and even more preferably between 1.5-5% by weight. The agar plate is then illuminated with fluorescent light at a relatively low light intensity. By a relatively low light intensity it is meant a light intensity that is from 1-50 μmol photon m⁻² s⁻², preferably from 5-20 μmol photon m⁻² s⁻², more preferably from 5-15 μmol photon m⁻² s⁻² and most preferably from 8-10 μmol photon m⁻² s⁻².

Biologically pure microcolonies can be grown and reproduced by transferring colonies from agar plates to an identical or different growth medium, preferably the identical growth medium, such as Algaen I as appears in Table 1 in a quantity that is between 100 ml to 2 l, with a 200 ml vessel being preferred. The growth medium is illuminated with fluorescent light at a moderate intensity. By moderate intensity, it is meant that an intensity of 50-400 μmol photon m⁻² s⁻², preferably from 100-250 μmol photon m⁻² s⁻², more preferably from 150-250 and most preferably from 200-250 μmol photon m⁻² s⁻² is used. A biologically pure culture of a cyanobacterium strain can be used to cultivate large amounts of cyanobacterium, in particular those species from the genus Nostoc, more particularly, Nostoc commune. By large amounts of cyanobacteria, it is meant that any amount can be generated from 100 ml to 20 l, preferably amounts from 200 ml to 15 l, more preferably amounts from 500 ml to 10 l, even more preferably amounts from 1 l to 5 l and most preferably amounts from 2 l to 5 l can be generated. The culture may be bubbled with CO₂-enriched air to provide mixing.

Macrocolonies can be induced by transfer of microcolonies to a different medium such as Algaen III as indicated in Table 1 and illuminating the culture with fluorescence at a high intensity. By high intensity, it is meant that an intensity of 400-1000 μmol photon m⁻² s⁻¹ preferably from 450-600 μmol photon m⁻² s⁻², more preferably from 500-600 and most preferably from 500-550 μmol photon m⁻² s⁻² is used.

The culture may optionally be bubbled with CO₂-enriched air to provide mixing. The cultures are allowed to grow for between 2 days to 10 weeks, more preferably from 5 days to 5 weeks, more preferably from 1 week to 5 weeks. After a week, substantially all of the colonies are 3 mm or larger in diameter, after two weeks, 90% of the colonies have a diameter of 5 mm and after 4 weeks, 80% of colonies have a diameter that is at least 10 mm.

The above-generated colonies are advantageous over those that are isolated in large amounts from nature because they do not have contaminants present in them. Moreover, the cultivated colonies have other qualities that are not present in Nostoc isolated from nature. One such difference is that the wild Nostoc contains xylose, mannose, galactose, glucose, glucuronic acid, whereas the cultivated Nostoc if cultivated as above, has not only the above carbohydrates but additionally contains the monosaccharides rhamnose and fucose. Moreover, there is a greater amount of phycobiliproteins produced in the cultivated colonies than those colonies that occur in nature. Generally, in the cultures made in the instant invention, the amount of phycobiliproteins is on the order of 0.8 to 1.2% by weight, whereas in nature, the amount of phycobiliproteins is generally less than this with amounts of 0.3% to 1.0% being common. By manipulating growth conditions, the amount of phycobiliproteins can be raised to levels that are on the order of 1.5% by weight or more. Phycobiliproteins are believed to have health benefits due to there suspected antioxidant activity.

The bioreactor of the instant invention is now described with reference to the figures. In addition to the letters referencing the components of the bioreactor, the figures also include dimensional information on the sizes of the respective components. This dimensional information is merely illustrative in nature and is not to be construed as limiting the scope of the invention.

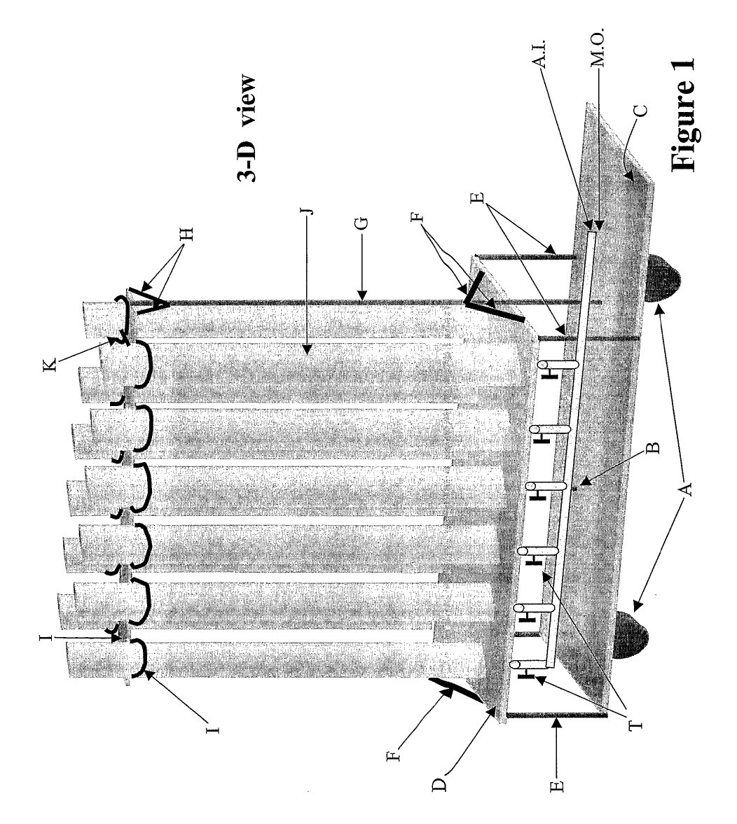

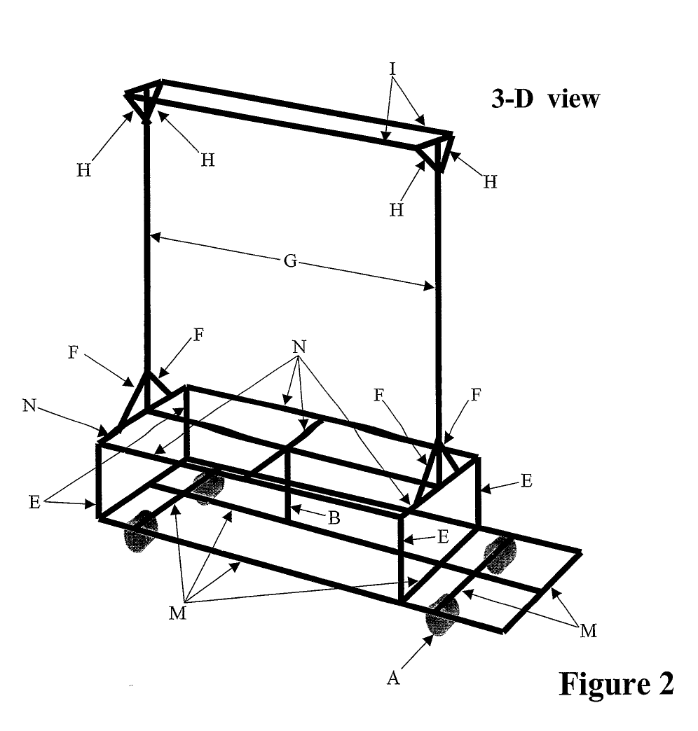

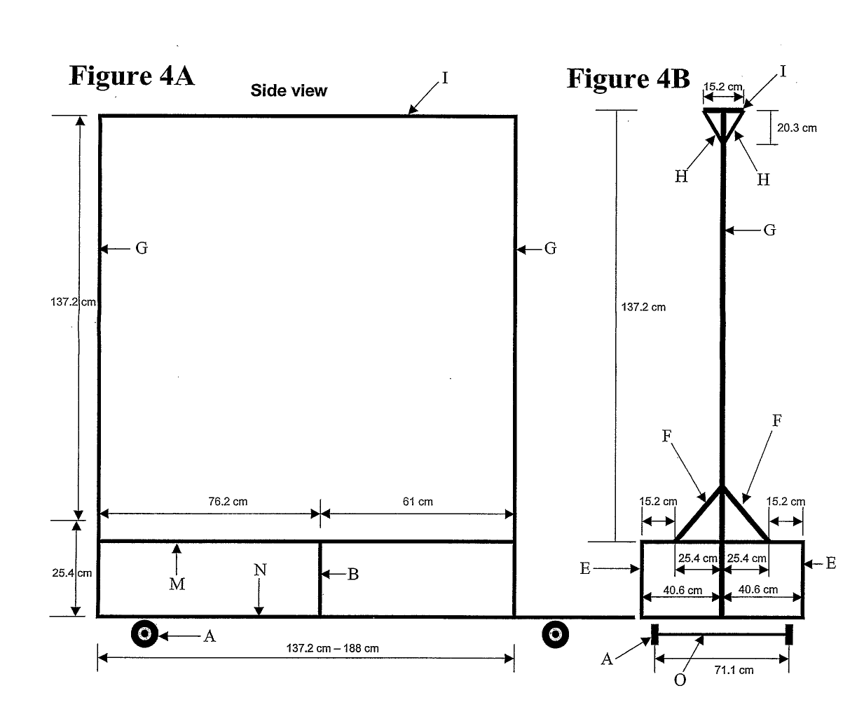

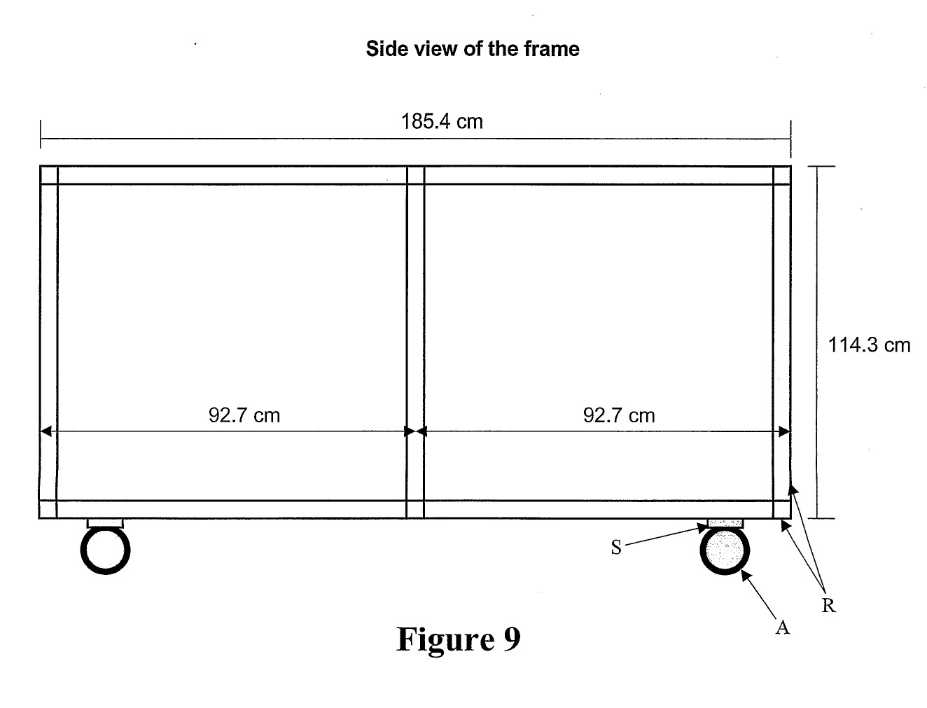

In one embodiment, the bioreactor of FIG. 1 is shown in 3-D. In FIG. 1, the bioreactor unit has three or more wheels A, which are attached to a baseboard C. The preferred configuration contains four wheels A although different numbers of wheels are within the scope of the invention. The wheels A can be either individually attached to the baseboard C wherein an axel is provided for each of the wheels by means that are readily known by those of skill in the art. Alternatively, two wheels can be attached to each other by means of an axel that goes from one wheel to another (please see wheel axel O in FIG. 4B for this option). Each wheel having its own axel is preferred. There is also included a means of attaching the wheels to the horizontal supporting rods M or the baseboard C (not shown in FIG. 1, however, see FIG. 9 for an example of an attaching means S to a frame). These attaching means are well known to those of skill in the art. On top of the baseboard C are vertical supporting rods E, with central vertical supporting rod B. Vertical supporting rods E and central vertical supporting rod B support the growth column supporting board D. On top of the growth column supporting board D are the growth columns J. On the baseboard C, there is attached an upright central vertical supporting rod G that is used to support the upper portion of the growth columns J. The central vertical supporting rod G is stabilized by brace rods F, which are in turn attached to growth column supporting board D. Attached to the central vertical supporting rod G are diagonal supporting rods H, which are in turn attached to top horizontal supporting rods I. It is understood that there can be one or more central vertical supporting rods G, with two central vertical supporting rods G being preferred. FIG. 1 does not show the second central vertical supporting rod G, although the two central vertical supporting rods G can be seen in FIG. 2. Top horizontal supporting rods I are positioned below or above the growth column connecting braces K, which stabilize the growth columns J relative to each other. The growth column connecting braces K are attached in turn to top horizontal supporting rods I. Connecting braces K are connected to supporting rings L, wherein supporting rings L are of a size that allow growth columns J to fit snugly inside.

In FIG. 1, the baseboard C is shown to be longer than the growth column supporting board D with the widths of each being flush on one end and the base board having overlap on the other end. It should be understood that the present invention encompasses the case where the dimensions of the growth column supporting board D and the baseboard C are the same so that there is no overlap. Moreover, included in the present invention is also the case wherein the growth column supporting board D is not flush with the width end of the baseboard C.

The growth columns can be glass, quartz, or any of a variety of plastics or any of a variety of polymer materials, with acrylic polymer materials being preferred. A consideration that should be kept in mind is the transparency of the material to fluorescent light. Because the cultures of cyanobacteria (or any bacteria, with Nostoc genus being preferred, and more particularly the Nostoc commune species being particularly preferred) are irradiated with fluorescent light, the material for the growth columns should be sufficiently transparent to the intensity and desired wavelength of transmitted light.

FIG. 1 also shows tubing for air input A.I. and for medium output M.O. that are regulated by the switches/valves T. These switches/valves T that are connected to the growth columns J and can be used to input any type of air with CO₂-enriched air being preferred and to output the media containing the culture.

FIG. 2 shows the rod frame network and the wheels A of the bioreactor, including the central vertical supporting rod B, vertical supporting rods E, brace rods F, central vertical supporting rods G, diagonal supporting rods H, and top horizontal supporting rods I. Moreover, the base board C as appears in FIG. 1 may be supported by a lattice of horizontal supporting rods M and the growth column supporting board D may be supported by a lattice of horizontal supporting rods N. A bioreactor that does not have the lattice of horizontal supporting rods M and the lattice of horizontal supporting rods N is contemplated and within the scope of the instant invention.

Any metal can be used for the rod frame network, including steel, iron, aluminum, various alloys of these metals, these metals as alloys with other metals, other metals used individually, and alloys of other metals, with steel being particularly preferred. Also contemplated are a wood rod frame network, and a plastic rod frame network. The limiting factors that determine the frame network are the structural integrity of the frame rods, i.e., whether the rods can support the weight of the bioreactor. Another factor that determines the frame network is whether the rods can successfully be attached together. For example, if steel is to be used as the frame network, the rods can be attached by soldering, by manufacture in a cast, attached by clamps, or by other means known to those of skill in the art. With plastic and wood rods, methods of attachment include screws, nails, nuts and bolts, hinges, and other means known to those of skill in the art. With these limitations in mind, the frame network can be created by just about any material known that satisfies these limitations.

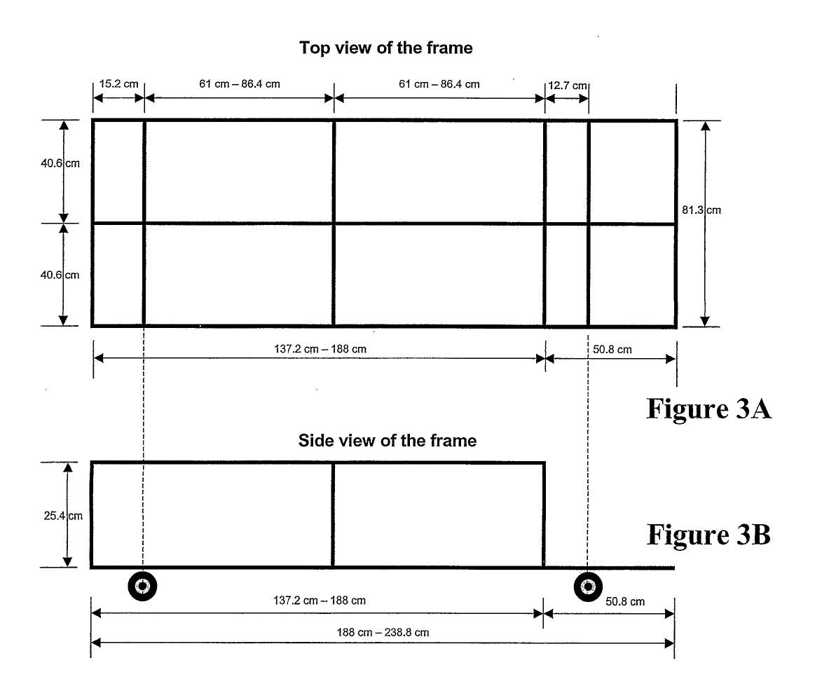

FIGS. 3A and 3B show a top view and a side view of the frame. The top view in FIG. 3A shows the horizontal supporting rods M and the lattice of horizontal supporting rods N. FIG. 3B shows the vertical supporting rods E, the central vertical supporting rod B, the horizontal supporting rods M, the lattice of horizontal supporting rods N, and the wheels A.

FIGS. 4A and 4B show a side view lengthwise view of the bioreactor and a side view widthwise of the bioreactor, respectively. FIG. 4a shows the vertical supporting rods E, the central vertical supporting rod B, the central vertical supporting rods G, the horizontal supporting rods M, the lattice of horizontal supporting rods N, top horizontal supporting rods I, and the wheels A. FIG. 4B shows a wheel axel 0, the wheels, A, the central vertical supporting rods G, the brace rods F, the diagonal supporting rods H, the vertical supporting rods E, the central vertical supporting rod B, and the top horizontal supporting rods I.

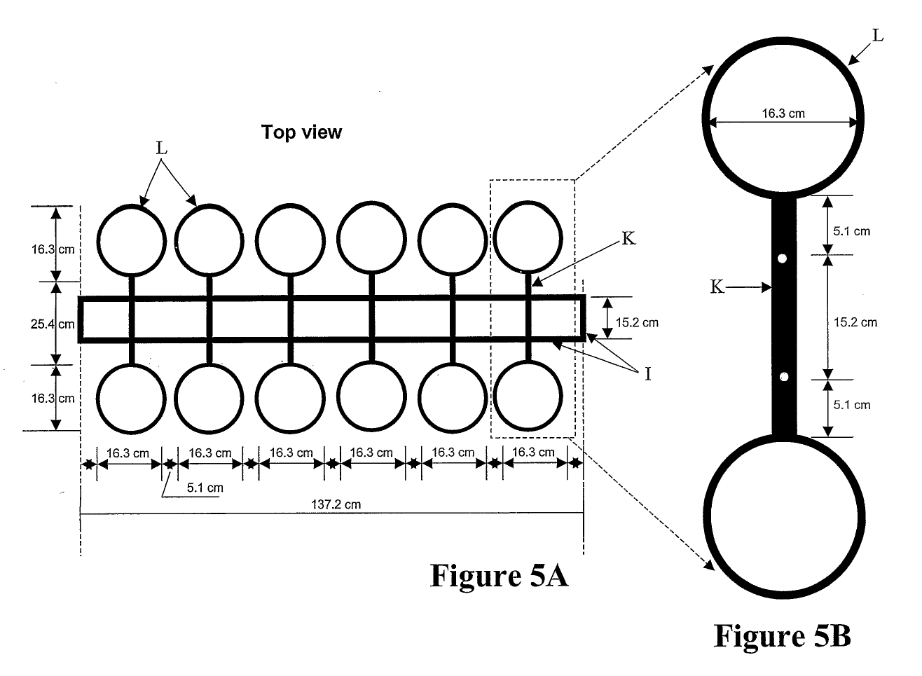

FIGS. 5A and 5B show a cross-sectional top view of the connecting braces K and the supporting rings L, and a blow-up sectional top view of the connecting braces K and the supporting rings L, respectively. In FIG. 5A, the top horizontal supporting rods I are also shown. In FIG. 5A, the top horizontal supporting rods I are shown below the connecting braces K, however, it is within the scope of the instant invention if the top horizontal supporting rods I are positioned above the connecting braces K.

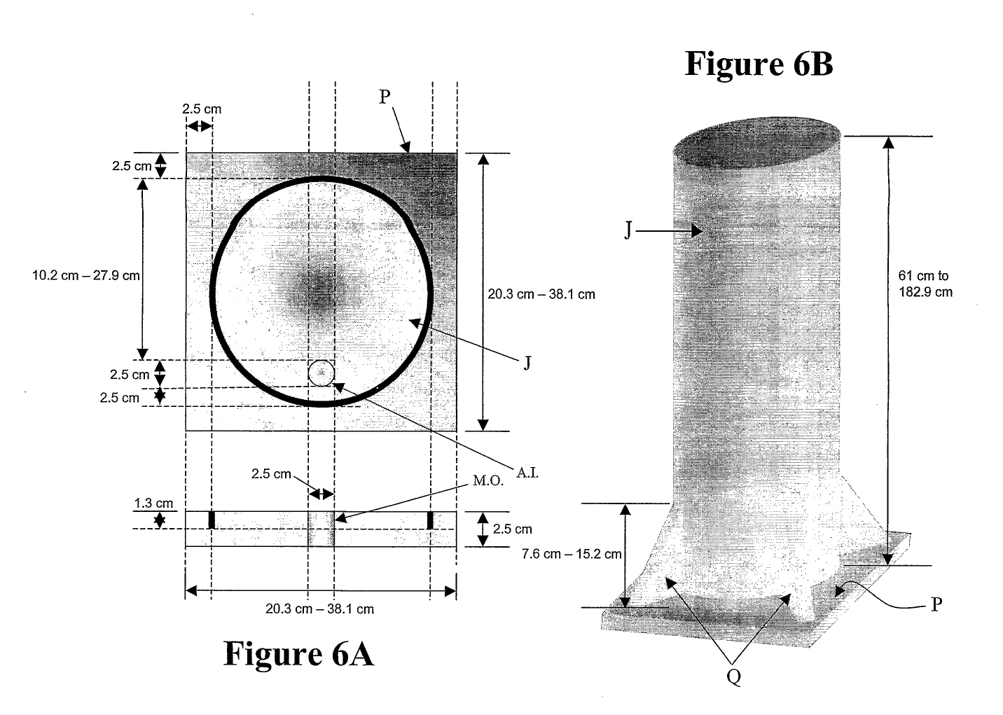

FIGS. 6A and 6B are a cross-sectional top view of one growth column J with an underlying base and a 3-D view of a growth column J with an underlying base, respectively. FIG. 6A also shows an air input A.I. that allows one to bubble CO₂ or any other gas. This air input can be equipped with a valve or without a valve. FIG. 6A also shows a medium output M.O. The underlying base P in FIG. 6A can be a polymer such as an acrylic based polymer, alternatively, wood or other materials known to those of skill in the art can be used. FIG. 6B shows a 3-D view of the growth column J with the base P made of a polymer such as an acrylic based polymer or any other material known to those of skill in the art. FIG. 6B also shows supporting triangles Q, which can be made out of a polymer such as acrylic-based polymers or any other material known to those of skill in the art.

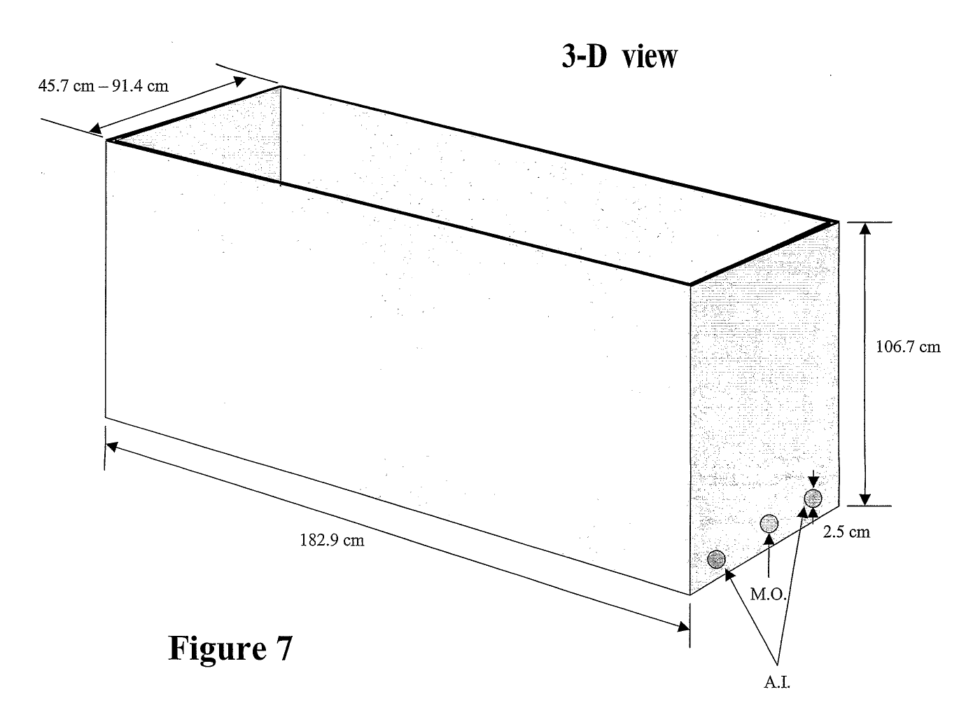

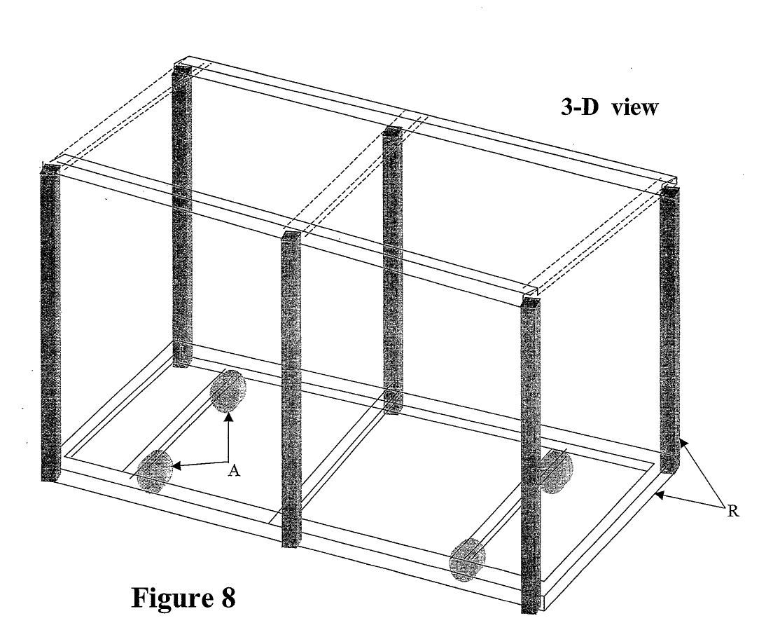

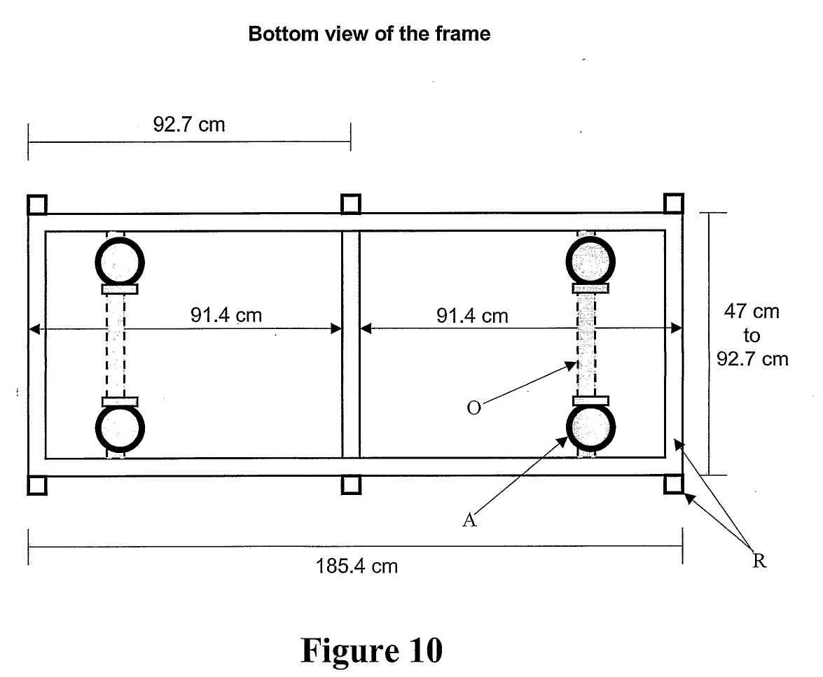

FIGS. 7-10 show another embodiment of the instant invention. In these figures, a tank that can be used for cultivation of large amounts of Nostoc is shown. In FIG. 7, a 3-D view of the tank is shown that can be used for cultivation. The tank material can be any material that is relatively transparent that allows the passage of fluorescent light, with glass, quartz, and plastics being possibilities, with glass being particularly preferred. I FIG. 7, there is also shown two air inlet ports A.I. and a medium outlet port M.O. It is within the scope of the instant invention to have one or more air inlet ports A.I. and one or more medium outlet ports, M.O. FIG. 8 shows a 3-D view of the frame R and the wheels A that supports the tank of FIG. 7. Any material that is strong enough to support a tank full of media can be used as the frame. A steel frame is the particularly preferred frame material. FIG. 9 shows the a side view of the frame that supports the tank of FIG. 7. FIG. 10 shows the bottom view of the frame that supports the tank of FIG. 7. It is within the scope of the instant invention to make minor modifications of the above described Figures.

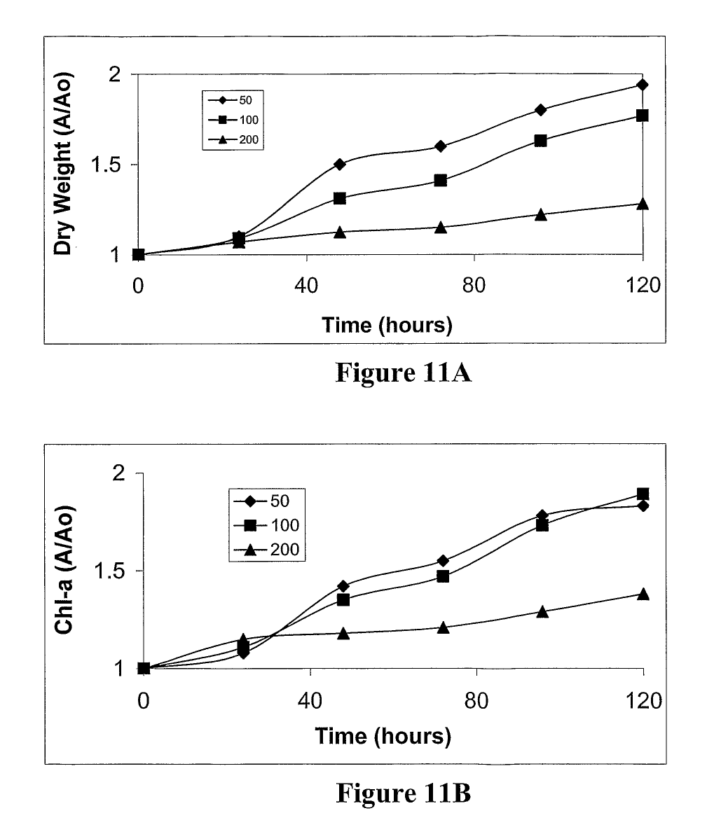

FIGS. 11A and 11B show the effect of light intensity (50, 100, or 200 mmol m⁻² s⁻¹) on the growth of Nostoc commune in Dry Weight (FIG. 11A) and in Chl-a (chloroform-a concentration (FIG. 11B)). The initial dry weight was 0.4 g/L, the initial Chl-a concentration was 5.3 mg/L. The data represent the average of two experiments.

Micro-colonies were incubated in a 1-liter glass bottle containing 800 ml Algaen I medium under three different light intensity levels (i.e., 50, 100, or 200 μmol m⁻² s⁻¹) at room temperature. The growth of the colonies was measured as increments in dry weight (DWt) as well as chlorophyll-a concentration (Chl-a). In the experiment shown in FIG. 11A, 50 μmol m⁻² s⁻¹ is an optimal light intensity, resulting in a maximal formation of mature macrocolonies. The growth rate decreased as light intensity increased to 100 and 200 μmol m⁻² s⁻¹ (see FIG. 11A).

In another embodiment, the present invention provides an edible Nostoc commune formulation, which includes fresh biomass or dried powder of Nostoc commune. Nostoc can be dried by any method that is known to those of skill in the art including air drying the culture. Alternatively, blowing air or vacuum drying are suitable alternatives for drying the Nostoc. In another aspect, the present invention provides a Nostoc commune formulation comprising one or any combination of proteins, fatty acids, polysaccharides and/or pigments.

The Nostoc commune formulation of the present invention may take many forms. For example, the Nostoc commune formulations of the present invention may be in powder form. Alternatively, the Nostoc commune formulations may be in tablet, capsule, or liquid form. In addition, the Nostoc commune formulations of the present invention may be included within a dietary supplement, or within food items, such as nutrition bars, liquid drinks, cereals, dairy products, etc. and in a food product of the present invention.

The Nostoc commune formulation of the present invention may be utilized in dietary supplements. In one aspect, a dietary supplement of the present invention comprises the Nostoc commune formulation of the present invention. A serving of a dietary supplement of the present invention could comprise 1 to 10 gram of a Nostoc commune formulation of the present invention.

A dietary supplement of the present invention may be in any digestible form, including a powder, a tablet, a capsule or in liquid form. A dietary supplement of the present invention may also be agglomerated and/or otherwise treated to improve solubility, digestibility or other aspects of the dietary supplement.

As will be understood by those of ordinary skill in the art, a dietary supplement of the present invention may also one or any combination of the ingredients selected from the group of herbals or herbal extracts, algal biomass, algal biomass extracts, fungi extracts, enzymes, a fiber source, minerals, vitamins and the like.

The present invention provides a digestible food product, which includes fresh or dried biomass of Nostoc commune. The food product of the present invention comprises a Nostoc commune formulation which can additionally contain one or any combination of proteins, fatty acids, polysaccharides and pigments.

The food product may further comprise additional components including any one or combination of preservatives, flavorings, vitamins, minerals and the like, including but not limited to calcium phosphate, soy lecithin, salt, potassium, chloride, artificial and natural flavors, carragenenan, carboxymethylcellulose, xanthan gum, water or milk. Among the carbohydrates that are suitable for use in the present invention include sucrose, fructose, glucose, galactose, lactose, mannose, mannitol, dextrose, maltodextrin, sorbitol, corn syrup solids, and other carbohydrates known to those of skill in the art.

A food product of the present invention may also be produced in a lower calorie form by substituting an artificial sweetener for all or a portion of the sugars. Suitable artificial sweeteners include sucralose (SPLENDA™), aspartame, saccharin and SINETK. (acesulfurame K). Plant derived sweeteners such as stevia are also suitable.

A food product of the present invention may take many forms, including a powder for dispersing in a liquid, a tablet, a bar, liquid drinks, a cereal, a dairy product, and the like. A food product of the present invention may further include any one or combination of ingredients selected from the group herbals, herbal extracts, algal biomass, algal biomass extracts, fungi extracts, enzymes, a fiber source, minerals, vitamins and the like.

Moreover, the present invention provides a pharmacological and/or a pharmaceutical composition comprising a Nostoc formulation of the present invention, with a Nostoc commune being particularly preferred. It should be understood that it may be the Nostoc that is the pharmaceutical agent or another agent that acts as the pharmaceutical agent. The Nostoc and the other agent may work independently and/or synergistically. Thus, the present invention provides a pharmacological and/or a pharmaceutical composition comprising a Nostoc commune formulation of the present invention and further comprising a medicinal composition. Suitable medicinal compositions include, but are not limited to the medicinal compositions, drugs and/or prescription drugs utilized in cholesterol lowering therapy, bone strengthening therapy, endometrial therapy, cancer therapy, including chemotherapy, Alzheimer's therapy, ulcer therapy, prostrate therapy, skin therapy, renal therapy, blood therapy, lymphatic therapy, lung therapy, nervous system therapy, diabetes therapy, eye therapy and the like. These medicinal compositions include, but are not limited to, Premarin, Fosamax, Raloxifene, Tamoxifen, SERM's (selective estrogen receptor modulators) and other drugs known to those of ordinary skill in the art.

An advantage of a pharmacological composition of the present invention comprising a Nostoc commune formulation of the present invention and a medicinal composition is that the combination may have synergistic effects. Therefore it may be possible to use a lesser amount of the medicinal composition in a pharmacological composition of the present invention than the amount traditionally utilized in the absence of a Nostoc commune formulation of the present invention, while achieving substantially the same desired therapeutic effects. This feature also may provide an additional advantage of reducing side or adverse effects caused by the medicinal composition due to the lower amount of the medicinal composition utilized.

The present invention also discloses a method for promoting the health of an individual comprising having the individual ingest greater than 50 grams fresh Nostoc commune or equivalent dried biomass, preferably greater than 100 grams fresh Nostoc commune or equivalent dried biomass, per day.

The present invention also provides methods for promoting and/or enhancing health which include digesting a Nostoc commune formulation of the present invention, and/or dietary supplements and/or food items and/or pharmacological compositions which include a Nostoc commune formulation of the present invention.

Section 1. Methodology for Strain Isolation and Purification

Colonies of Nostoc commune were collected from the Yadkin River in Forsyth County, N.C. during the spring. After washing with sterile Algaen-I medium, the colonies were crushed with a mortar and pestle, the cells were spread onto an agar plate containing Algaen-I and the plates were illuminated with fluorescent light. After one week, the cells from the plates were transferred to a fresh plate. After three transfers, axenic colonies were obtained, which were used for further cultivation.

Section 2. Steps for Cultivating Nostoc commune:

The cultivation of Nostoc commune comprises the following steps:

2a. Hormogonia generation in Algaen-II

2b. Formation of microcolonies on an agar plate containing Algaen-III medium

2c. Growth and reproduction of microcolonies in culture vessels

2d. Formation of macrocolonies in culture vessels.

Section 3. Optimal Conditions for Each of the Cultivating Steps Above.

3a. Hormogonia generation:

To induce hormogonia generation, macrocolonies of Nostoc commune were washed three times with Algaen-II medium as described in section 2. The washed macrocolonies were re-suspended in Algaen-II medium for 3 days at 25° C. with illumination with 100 μmol photon m⁻² s⁻¹ light, whereupon hormogonia were released from the colonies.

Hormogonia may also be obtained by grinding macrocolonies with a mortar and pestle in Algaen-I medium.

3b. Formation of microcolonies on agar plates:

Hormogonia were resuspended in Algaen-I medium and spread on an agar plate containing 1.5% agar and Algaen-I medium at a concentration of 1000 cells/plate. The plates were sealed with parafilm and incubated at 25° C. with illumination by 10 μmol photon m⁻² s⁻² light. After one week, the formation of microcolonies was observed with a microscope. After three weeks, the microcolonies were ready for transfer to a liquid growth medium.

3c. Growth and reproduction of microcolonies in culture vessels

The microcolonies obtained from the agar plates described above were transferred to a culturing vessel containing 200 ml Algaen-I medium. The culture was illuminated with fluorescent bulbs at a light intensity of 200 μmol photon m^{-2.}s⁻². The culture was mixed by bubbling the liquid with CO₂-enriched air. The culturing vessels can be glass bottles, transparent plastic bottles or other transparent containers. Although the instant case used 200 ml cultures, the volume of acceptable culturing vessels is from 100 ml to 20 liter.

3d. Formation of macrocolonies in culturing vessels

To induce the formation of macrocolonies, microcolonies were transferred to a medium under the following conditions: (a). Algaen-III medium as described in Section 2; (b). Light intensity of 500 μmol photon m⁻² s⁻¹. Under these conditions, microcolonies stopped division and reproduction, instead, all microcolonies continuously grew to increase their volume. The diameter increased from less than 1 mm to more than 3 mm after one week. After two weeks, 90% of the colonies reached a diameter of 5 mm. After four weeks, about 80% of the colonies reached a diameter of 10 nun.

While the invention has been described with reference to numerous specific details, one of ordinary skill in the art will recognize that the invention can be embodied in other specific forms without departing from the spirit of the invention. Thus, one of ordinary skill in the art would understand that the invention is not to be limited by the foregoing illustrative details, but rather is to be defined by the appended claims. Moreover, it is contemplated that the instant invention encompasses any limitation from any claim in combination with any one or more limitation from any other claim.

The following recited references are incorporated by reference in their entirety.

### REFERENCES CITED

Aki B., Heilmann J. and Sticher O. (2000) New antibacterial metabolites from the cyanobacterium Nostoc commune (EAWAG 122b). J Nat. Prod. 63: 1283-1285.
Ang-Lee M. K. Moss J. and Yuan C. S. (2001) Herbal medicines and perioperative care. JAMA 286: 208-216.
Brown M. R. (1991) The amino acid and sugar composition of 16 microalgae used in mariculture. J. Exp. Mar. Biol. Ecol. 145: 79-99.
Brown M. R. and Jeffery S. W. (1995) The amino acid and gross composition of marine diatoms potentially useful for mariculture. J. Appl. Phycol. 7: 521-527.
Esser M. T., Mori T., Mondor I., Sattentau Q. J., Dey B., Berger E. A., Boyd M. R., and Lifson J. D. (1999) Cyanovirin-N binds to gp120 to interfere with CD4-dependent human immunodeficiency virus type 1 virion binding, fusion, and infectivity but does not affect the CD4 binding site on gp120 or soluble CD4-induced conformational changes in gp120. J. Virol. 73(5): 4360-71.
Golakoti T., Ogino J., Heltzel C. E., Le H. T., Jensen C. M., Larsen L. K., Patterson G. M. L., Moore R. E., Mooberry S. L., et al. (1995) Structure determination, conformational analysis, chemical stability studies, and antitumor evaluation of the cryptophycins. Isolation of 18 new analogs from Nostoc sp. strain GSV 224. Journal of the American Chemical Society 117, no. 49: 12030-49.
Gonzalez R., Rodriguez S., Romay C., Ancheta O., Gonzalez A., Annesto J., Remirez D., and Merino N. (1999) Anti-inflammatory activity of phycocyanin extract in acetic acid-induced colitis in rats. Pharmacol Res. 39(1): 55-9.
Hayashi K., Hayashi T., and Kojima I. (1996) AIDS res. Human Retroviruses 12: 1463-71.
Hori K. G., Ishibashi G., et al. (1994) Hypocholesterolemic effect of blue-green alga, ishikurage (Nostoc commune) in rats fed atherogenic diet. Plant Foods Hum Nutr. 45: 63-70.
Hori K., Ueno-Mohri T., Okita T., and Ishibashi G. (1990) Chemical composition, in vitro protein digestibility and in vitro available iron of blue green alga, Nostoc commune. Plant Foods Hum Nutr. 40(3): 223-9.
Hu Q., Guterman H. and Richmond A. (1996) A flat inclined modular photobioreactor (FIMP) for outdoor mass cultivation of photoautotrophs. Biotechnol. Bioeng. 51: 51-60.
Huang Z. B., Liu Y. D., Paulsen B. S., Klaveness D. (1998) Studies on polysaccharides from three edible species of Nostoc (Cyanobacteria) with different colony morphologies: Comparison of monosaccharide compositions and viscosities of polysaccharides from field colonies and suspension cultures. Journal Of Phycology 34 (6): 962-968.
Knubel G., Larsen L. K., Moore R. E., Levine I. A., and Patterson G. M. (1990) Cytotoxic, antiviral indolocarbazoles from a blue-green alga belonging to the Nostocaceae. J Antibiot (Tokyo) 43(10): 1236-9.
Komarek J. and Anagnostidis K. (1989) Modern approach to the classification system of cyanophytes. 4-Nostocales. Arch. Hydrobiol. Suppl. 823, Algological Studies 56: 247-345.
Murakami M., Sun Q., Ishida K., Matsuda H., Okino T., and Yamaguchi K. (1997) Microviridins, elastase inhibitors from the cyanobacterium Nostoc minutum (NIES-26) Phytochemistry (Oxford), 45(6): 1197-1202
Philippis R. D. and Vincenzini M. (1998) Exocellular polysaccharides from cyanobacteria and their possible applications. FEMS Microbiol. Rev. 22: 151-175.
Piccardi R., Frosini A., Tredici M. R., and Margheri M. C. (2000) Bioactivity in free-living and symbiotic cyanobacteria of the genus Nostoc. J Appl. Phycol. 12: 543-547.
Qiu B. S., Liu J. Y., Liu Z. L., Liu S. X. (2002) Distribution and ecology of the edible cyanobacterium Ge-Xian-Mi (Nostoc) in rice fields of Hefeng County in China: Journal Of Applied Phycology 14 (5): 423-429.
Scherer S. and Potts M. (1989) Novel water stress protein from a desiccation-tolerant cyanobacterium: purification and partial characterization. J. Biol. Chem. 264: 12546-12553.
Smith C. D., Zhang X., Mooberry S. L., Patterson G. M., and Moore R. E. (1994) Cryptophycin: a new antimicrotubule agent active against drug-resistant cells. Cancer Res. 54(14): 3779-84.
Takenaka H., Yamaguchi Y., Sakaki S., Watarai K., Tanaka N., Hori M., Seki H., Tsuchida M., Yamada A., Nishimori T., and Morinaga T. (1998) Safety evaluation of Nostoc flagelliforme (nostocales, cyanophyceae) as a potential food. Food-and-Chemical-Toxicology. 36(12): 1073-1077.
Wolk C. P. (1996) Heterocyst formation. Ann. Rev. Genet. 30: 59-78.

We claim:

1. A biologically pure culture of a cyanobacterium strain belonging to the genus Nostoc, wherein the culture comprises colonies.

2. The culture according to claim 1, wherein the species is Nostoc commune.

3. The culture according to claim 1, wherein substantially all of the Nostoc colonies are of a diameter that is between about 3 mm and about 5 mm.

4. The culture according to claim 1, wherein said culture is substantially free of contaminants, wherein the contaminants are selected from the group consisting of pesticides, fungicides, insecticides and herbicides.

5. The culture according to claim 3, wherein the average diameter of said colonies are about 4 mm.

6. A method of producing a biologically pure culture of a cyanobacterium strain belonging to the genus Nostoc, wherein said method comprises:
a) crushing a Nostoc colony to generate a crushed Nostoc colony,
b) spreading said crushed Nostoc colony onto an agar plate,
c) illuminating said agar plate containing said Nostoc colony with fluorescent light,
d) transferring said Nostoc colony to a fresh agar plate,
to produce a biologically pure culture of Nostoc.

7. The method according to claim 6, further comprising
e) repeating step d) from 1 to 3 times

8. The method according to claim 6, wherein the culture is Nostoc commune.

9. A method of cultivating colonies of Nostoc comprising:
a) generating Nostoc hormogonia in a growth medium,
b) illuminating the Nostoc with fluorescent light,
c) spreading the illuminated Nostoc microcolonies on an agar plate to generate Nostoc microcolonies,
cultivating the microcolonies of Nostoc.

10. The method according to claim 9, further comprising the steps:
d)transferring the microcolonies to a growth medium identical to or different from said growth medium in step a),
e) illuminating the microcolonies in the growth medium with fluorescent light to generate microcolonies of Nostoc,
cultivating the microcolonies of Nostoc.

11. The method according to claim 9, further comprising the steps:
d) transferring the microcolonies to a growth medium identical or different to said growth medium in step a);
e) applying a fluorescent light intensity that is at least 400 μmol photon m⁻² s⁻¹ to generate macromolecules;
cultivating the macrocolonies of Nostoc.

12. The method according to claim 10, further comprising bubbling the microcolonies in the liquid growth medium that is identical of different from the growth medium in step a) with CO₂.

13. The method according to claim 9, wherein the colonies are Nostoc commune.

14. The method according to claim 11, further comprising bubbling the microcolonies in the liquid growth medium that is identical or different from the growth medium in step a) with CO₂.

15. The method according to claim 13, wherein at least 100 ml of the liquid growth medium is used.

16. The method according to claim 13, wherein substantially all of the macrocolonies are at least about 5 mm in diameter.

17. The method according to claim 16, wherein about 80% of the colonies are at least about 10 mm in diameter.

18. The method according to claim 10, wherein the growth medium in step d) is different.

19. The method according to claim 11, wherein the growth medium in step d) is different.

20. The method according to claim 10, wherein the growth medium in step d) is identical.

21. The method according to claim 11, wherein the growth medium in step d) is identical.

22. A composition comprising a cyanobacterium strain belonging to the genus Nostoc, wherein the composition comprises Nostoc colonies with an acceptable diluent, excipient, or carrier.

23. The composition according to claim 22, wherein the Nostoc is Nostoc commune.

24. The composition according to claim 22, wherein the Nostoc are substantially uniform in size and of a diameter that is between about 3 mm and about 5 mm.

25. The composition according to claim 22, wherein the composition is substantially free of contaminants, wherein the contaminants are selected from the group consisting of pesticides, fungicides, insecticides and herbicides.

26. The composition according to claim 22, wherein the composition further comprises medicine.

27. A pharmaceutical composition comprising a pharmaceutically effective amount of a cyanobacterium strain belonging to the genus Nostoc, wherein the composition comprises colonies along with a pharmaceutically acceptable diluent, excipient, or carrier.

28. The pharmaceutical composition according to claim 27, wherein the Nostoc colonies are substantially uniform and have a diameter of between about 3 mm and about 5 mm.

29. The pharmaceutical composition according to claim 27, wherein the composition comprises Nostoc and a medicine wherein the medicine is the pharmaceutically active ingredient.

30. The pharmaceutical composition according to claim 27, wherein the pharmaceutical composition contains Nostoc commune.

31. The pharmaceutical composition according to claim 30, wherein the composition comprises powdered Nostoc commune.

32. The pharmaceutical composition according to claim 27, wherein the composition is made by a process comprising the steps of:
a) collecting a Nostoc colony from a natural source,
b) washing said Nostoc colony with a sterile medium,
c) crushing said Nostoc colony,
d) spreading said Nostoc colony onto an agar plate,
e) illuminating said agar plate containing said Nostoc colony with fluorescent light,
f) transferring said Nostoc colony to a fresh agar plate,
g) repeating step f) from 1 to 3 times to produce a biologically pure culture of Nostoc
and combining said biologically pure culture of Nostoc with a pharmaceutically acceptable diluent, excipient, or carrier.

33. The pharmaceutical composition according to claim 32 wherein the Nostoc is Nostoc commune.

34. The pharmaceutical composition according to claim 27, wherein the pharmaceutical composition further comprises a medicine for treating a disorder selected from the group consisting of an anti-viral medicine, a cholesterol lowering medicine, and an anti-inflammatory medicine.

35. The pharmaceutical composition according to claim 34, wherein the medicine is selected from the group consisting of Arthrotec, Asacol, Auralgan, Azulfidine, Bextra, Celestone, Daypro, Deltasone, Diclofenac, Etodolac, Indocin, Ketoprofen, Lodine, Mobic, Nabumetone, Naproxen, Piroxicam, Ponstan, Prednisone, Rofecoxib, Salofalk, Solumedrol, Premarin, Fosamax, Raloxifene, Tamoxifen, Tamoxifen citrate, casodex, hydrea, mercaptopurine, methotrexate lederle, and SERM's.

36. A cyanobacterial growing medium comprising water, sodium nitrate, sodium carbonate, EDTA, citric acid, ferric ammonium citrate, and manganese chloride and one or more members selected from the group consisting of magnesium sulfate, calcium chloride, dibasic potassium phosphate, cobalt nitrate, copper sulfate, sodium molybdate, boric acid, and zinc sulfate.

37. The medium according to claim 36 wherein in the medium sodium nitrate is present in an amount of from 1.5 to 2.0 g/L, sodium carbonate is present in an amount of from 0.02 to 0.05 g/L, EDTA is present in an amount of from 0.001 to 0.003 g/L, citric acid is present in an amount of from 0.006 to 0.012 g/L, ferric ammonium citrate is present in an amount of from 0.006 to 0.012 g/L, and manganese chloride is present in an amount of from 1.81 to 3.62 ppm.

38. The medium according to claim 37, wherein the medium contains one or more members selected from the group consisting of magnesium sulfate, calcium chloride, dibasic potassium phosphate, cobalt nitrate, copper sulfate, sodium molybdate, boric acid, and zinc sulfate wherein the magnesium sulfate, if present, is present in an amount of from 0.015 to 0.15 g/L, calcium chloride, if present, is present in an amount of from 0.1 to 0.54 g/L, dibasic potassium phosphate, if present, is present in an amount of from 0.01 to 0.075 g/L, cobalt nitrate, if present, is present in an amount of from 0.049 to 0.17 ppm, copper sulfate, if present, is present in an amount of from 0.079 to 0.3 ppm, sodium molybdate, if present, is present in an amount of from 0.039 to 1.2 ppm, boric acid, if present, is present in an amount of from 2.86 to 12.4 ppm, and zinc sulfate, if present, is present in an amount of from 0.05 to 0.10 ppm.

39. The medium according to claim 38, wherein the medium contains all of the members selected from the group consisting of magnesium sulfate, calcium chloride, dibasic potassium phosphate, cobalt nitrate, copper sulfate, sodium molybdate, boric acid, and zinc sulfate.

40. A bioreactor comprising three or more wheels, a frame, and one or more containers for cultivating bacteria, wherein the wheels allow the bioreactor to be moved, the wheels support the frame and the frame supports the one or more containers for cultivating bacteria and the one or more containers are made of a material that allows the passage of fluorescent light.

41. A food product comprising a biologically pure culture of a cyanobacterium strain belonging to the genus Nostoc.

42. The food product according to claim 41, wherein the species is Nostoc commune.

43. The food product according to claim 41 wherein Nostoc is present in an amount that is at least 50 grams.

44. The food product according to claim 42, wherein Nostoc commune is present in an amount that is at least 50 grams.

45. A method for promoting or enhancing health comprising administering to an individual in need of health promotion or enhancement a health promoting or enhancing amount of a composition comprising a biologically pure culture of a cyanobacterium strain belonging to the genus Nostoc.

46. The method according to claim 45, wherein the Nostoc strain is Nostoc commune.

47. A dietary supplement comprising a biologically pure culture of a cyanobacterium strain belonging to the genus Nostoc.

48. The dietary supplement of claim 47, wherein the Nostoc is Nostoc commune.

49. The dietary supplement according to claim 48, wherein the supplement is in the form of a capsule or tablet.

50. The dietary supplement according to claim 48, wherein the supplement is in a powdered form.
